(19) United States
(12) Patent Application Publication
(10) Pub. No.: US 2026/0319036 A1
(43) Pub. Date: Oct. 15, 2026

(54) RECEIVING COIL DEVICE AND MAGNETIC RESONANCE-GUIDED RADIOTHERAPY APPARATUS

(71) Applicant: Shanghai United Imaging Healthcare Co., Ltd., Shanghai (CN)

(72) Inventors: Xiangbin Fang, Shanghai (CN); Haiyu Xie, Shanghai (CN); Shao Che, Shanghai (CN); Junjun Qin, Shanghai (CN)

(21) Appl. No.: 19/645,405

(22) Filed: Apr. 13, 2026

(30) Foreign Application Priority Data
Apr. 11, 2025 (CN) 202510462051.4

(51) Int. Cl. G01R 33/3415; A61N 5/10

(52) U.S. Cl. CPC G01R 33/3415; A61N 5/1049; A61N 2005/1055

(57) ABSTRACT

The present disclosure relates to a receiving coil device and a magnetic resonance-guided radiotherapy apparatus. The receiving coil device is applicable to magnetic resonance-guided radiation delivery, and the receiving coil device includes: a plurality of coil units, including at least one first coil unit in a first region and at least one second coil unit in a second region. The at least one first coil unit is arranged along a first direction, and the at least one second coil unit is arranged along the first direction. The first region and the second region are arranged along a second direction, and the first direction is perpendicular to the second direction.

### CROSS-REFERENCE TO RELATED APPLICATIONS

The present disclosure claims priority to Chinese patent application No. 202510462051.4, filed on Apr. 11, 2025, and entitled “Receiving Coil Device and Magnetic resonance-guided Radiotherapy Apparatus”, the entire content of which is incorporated herein by reference.

### TECHNICAL FIELD

The present disclosure relates to the technical field of magnetic resonance imaging, and in particular, to a receiving coil device and a magnetic resonance-guided radiotherapy apparatus.

### BACKGROUND

In the design of magnetic resonance-guided radiotherapy apparatuses, to meet the real-time magnetic resonance imaging requirements during coplanar radiotherapy, a magnetic resonance imaging apparatus is combined with a radiotherapy apparatus, so that during radiotherapy, real-time scanning and imaging of a patient’s treatment site can be performed.

However, the applicant has found that in conventional solutions, as the apparatus is used over time, the magnetic resonance imaging performance becomes unstable, making it difficult to provide reliable real-time magnetic resonance images for radiotherapy, thereby affecting the effectiveness of radiotherapy.

### SUMMARY

In a first aspect, a receiving coil device is provided, applicable to magnetic resonance-guided radiation delivery. The receiving coil device includes: a first coil unit, at least a portion of the first coil unit being disposed in a radiation region, the radiation region being irradiated by radiation with a dose at or above a threshold during a radiation delivery, and a second coil unit, at least a portion of the second coil unit being disposed in a non-radiation region outside the radiation region.

In a second aspect, a receiving coil device is further provided, applicable to magnetic resonance-guided radiation delivery. The receiving coil device includes: a plurality of coil units, including at least one first coil unit in a first region and at least one second coil unit in a second region. The at least one first coil unit is arranged along a first direction, the at least one second coil unit is arranged along the first direction, and the first region and the second region are arranged along a second direction. The first direction is perpendicular to the second direction.

In a third aspect, a receiving coil device is further provided, applicable to magnetic resonance-guided radiation delivery. The receiving coil device includes: a first region, the first region including at least one first coil unit arranged along a first direction; a second region, the second region including at least one second coil unit arranged along the first direction; and an electronic component disposed in a region other than the first region and electrically connected to the at least one first coil unit and/or the at least one second coil unit.

In a fourth aspect, a magnetic resonance-guided radiotherapy apparatus is provided, including the receiving coil device as described above.

In a fifth aspect, a magnetic resonance-guided radiation delivery method is provided, including: obtaining, through a first region of a receiving coil device, a magnetic resonance signal of a target site of a target object, and generating a first image, the first image being configured for radiation delivery; and obtaining, through a second region of the receiving coil device, a magnetic resonance signal of a non-target site of the target object, and generating a second image, the second image being configured for monitoring the target object.

In a sixth aspect, an image display method is provided, applicable to magnetic resonance-guided radiation delivery. The image display method includes: displaying a first image to a user during a first period within the radiation delivery, the first image including a target region of the radiation delivery; and displaying a second image to a user during a second period within the radiation delivery, before the radiation delivery, and/or after the radiation delivery, the second image including a non-target region of the radiation delivery.

In a seventh aspect, a non-transitory computer-readable storage medium is provided, having a computer program stored thereon. The computer program, when executed by a processor, implements the steps of the method according to the fifth aspect or the sixth aspect.

### BRIEF DESCRIPTION OF THE DRAWINGS

To more clearly describe the technical solutions in the embodiments of the present disclosure or in the conventional technology, the accompanying drawings required for describing the embodiments or the conventional technology will be briefly introduced below. Apparently, the accompanying drawings in the following description are merely some embodiments of the present disclosure. For a person of ordinary skill in the art, other drawings can be derived from these drawings without any creative effort.

FIG. 1 is a schematic structural diagram of a receiving coil device according to some embodiments.

FIG. 2 is a schematic structural diagram of a receiving coil device according to other embodiments.

FIG. 3 is a schematic structural diagram of a receiving coil device according to other embodiments.

FIG. 4 is a schematic flow diagram of a magnetic resonance-guided radiation delivery method according to some embodiments.

### DETAILED DESCRIPTION OF THE EMBODIMENTS

In order to facilitate understanding of the present disclosure, the present disclosure will be described more fully below with reference to the relevant accompanying drawings. Embodiments of the present disclosure are presented in the accompanying drawings. However, the present disclosure may be implemented in many different forms and is not limited to the embodiments described herein. Rather, these embodiments are provided for the purpose of making the present disclosure more thorough and comprehensive.

Unless otherwise defined, all technical and scientific terms used herein have the same meaning as commonly understood by a person of ordinary skill in the art to which the present disclosure belongs. The terms used herein in the specification of the application are for the purpose of describing specific embodiments only, and are not intended to limit the present disclosure.

It should be understood that the terms “first”, “second”, etc. used in this application may be used herein to describe various elements, but these elements are not limited by these terms. These terms are only used to distinguish the first element from another element. For example, without departing from the scope of the present disclosure, the first coil unit may be referred to as the second coil unit, and similarly, the second coil unit may be referred to as the first coil unit. Both the first coil unit and the second coil unit are coil units, but they are not the same coil unit.

It should be understood that “at least one” refers to one or more, while “a plurality of” refers to two or more. “At least part of an element” means part or all of an element.

As used herein, the singular forms “a”, “an” and “the” may also include the plural forms, unless the context clearly indicates otherwise. It should also be understood that the terms “includes/comprises” or “has” etc. designate the presence of stated features, integers, steps, operations, components, parts or combinations thereof, but do not preclude the possibility of the presence or addition of one or more other features, integers, steps, operations, components, parts or combinations thereof.

A magnetic resonance-guided radiotherapy apparatus mainly includes a main magnet system, a gradient magnetic field system, a radio frequency system, a computer processing system, and an auxiliary device. The function of the main magnet system is to provide a static magnetic field necessary for aligning atomic nuclei. The function of the gradient magnetic field system is to generate a spatially varying magnetic field using gradient coils and superimpose it on the main magnetic field, thereby spatially encoding magnetic resonance imaging signals. The radio frequency system transmits radio frequency pulses so that magnetized protons absorb energy and resonate, and receives magnetic resonance imaging signals (MRI signals) generated by the energy released during proton relaxation. The computer system may include, for example, a host, a memory, input/output devices, system and application software, and is used to process the received MRI signals for imaging, display, data processing, etc. The auxiliary device may include, for example, an operation console, an examination table, a high-pressure injector, etc. required during the examination process.

In the radio frequency system, a signal source excites a transmitting coil to transmit radio frequency signals, stimulating the human body to resonate. A radio frequency receiving coil receives magnetic resonance imaging signals generated during resonance of the human body and transmits the magnetic resonance imaging signals to a processing circuit which includes an electronic component. The processing circuit performs analog-to-digital conversion, decoding, digital signal processing, etc. on the received magnetic resonance imaging signals to generate magnetic resonance images.

A magnetic resonance-guided radiotherapy apparatus combines a magnetic resonance apparatus with a conventional radiotherapy apparatus, providing a basis for the radiotherapy process based on the magnetic resonance images, so as to improve the effectiveness of radiotherapy.

During treatment, it is necessary to meet the real-time magnetic resonance imaging requirements during coplanar radiotherapy.

However, the applicant has found that as an important component exposed to a radiation region, the radio frequency receiving coil contains many electronic components (e.g., capacitors, inductors, operational amplifiers) that are difficult to geometrically model. Therefore, the presence of the radio frequency receiving coil affects the planning of tumor radiotherapy plans and dose calculation, thus influencing the effectiveness of radiotherapy to some extent. For the radio frequency receiving coil, some radiation-sensitive electronic components (e.g., operational amplifier chips) are also damaged by radiation. As an exposure time to radiation increases, the service life of the components is shortened, affecting the performance of the radio frequency receiving coil, thereby affecting real-time magnetic resonance imaging and hindering timely adjustment of radiotherapy plans during treatment.

The present disclosure mainly relates to improvements to the radio frequency receiving part for magnetic resonance imaging, and in particular to improvements to the radio frequency receiving coil and the electronic component disposed thereon.

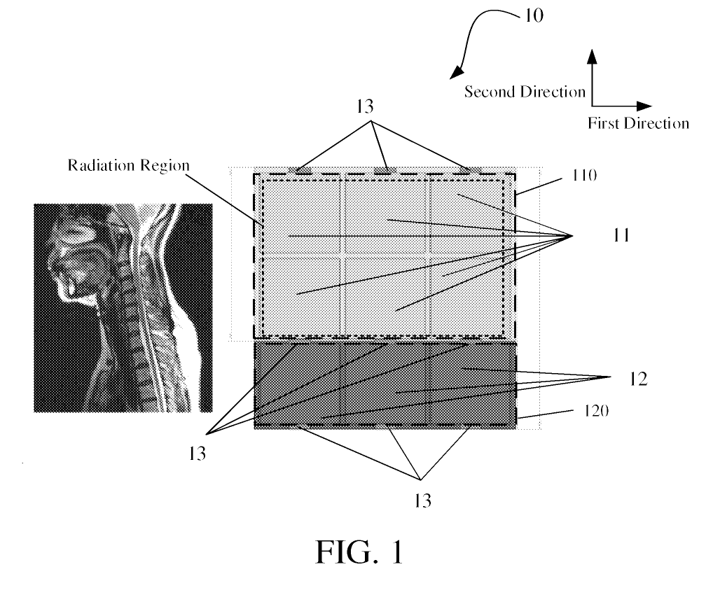

In an embodiment, as shown in FIG. 1, a receiving coil device 10 is provided, applied to a magnetic resonance-guided radiotherapy apparatus. The magnetic resonance-guided radiotherapy apparatus integrates the above-described magnetic resonance imaging apparatus, and can perform magnetic resonance imaging on a target object (e.g., a patient, a phantom, etc.) during radiotherapy. As an example, during treatment, an electron linear accelerator in the radiotherapy apparatus generates microwaves via a magnetron. The microwaves are fed into an accelerating tube cavity through a coupling waveguide, forming an electromagnetic field. An electron beam emitted by an electron gun is accelerated in the accelerating tube cavity and then strikes a target surface, generating high-energy X-rays (i.e., radiation). The X-rays pass through a beam limiting system (e.g., including a primary collimator, a secondary collimator, a multi-leaf collimator, etc.) to form a radiation field required for treatment, irradiating a target volume (e.g., a target volume of tumor of a patient, a specific region of a phantom, etc.) of the target object positioned on a support platform (e.g., a sickbed, a worktable, etc.) to perform radiotherapy.

As shown in FIG. 1, the receiving coil device 10 can be applied to magnetic resonance-guided radiation delivery. The receiving coil device 10 can include a plurality of coil units, and the plurality of coil units include at least one first coil unit 11 located in a first region 110 and at least one second coil unit 12 located in a second region 120. The at least one first coil unit 11 and the at least one second coil unit 12 are both arranged along a first direction. The first region 110 and the second region 120 are arranged along a second direction. The first direction is perpendicular to the second direction.

Exemplarily, the second direction may be set as a main magnetic field direction of the magnetic resonance (corresponding to a head-foot direction of the patient, also referred to as a longitudinal direction of the human body) of the magnetic resonance-guided radiotherapy apparatus, and the first direction may be set as a direction (corresponding to a direction perpendicular to the head-foot direction of the patient, also referred to as a transverse direction of the human body) perpendicular to the main magnetic field direction of the magnetic resonance of the radiotherapy apparatus. This arrangement can provide greater acceleration in the head-foot direction, making the magnetic field distribution more uniform in the longitudinal direction of the human body, thereby reducing image artifacts caused by inhomogeneity of the magnetic field.

As an example, the receiving coil device 10 may be a surface coil. As an optional example, the first coil units 11 and the second coil units 12 may be detachably or fixedly arranged on a support or frame of the receiving coil device 10. As an optional example, the first coil units 11 and the second coil units 12 may be assembled to form the receiving coil device 10. During assembly, the first coil units 11 and the second coil units 12 may be arranged spaced apart from each other, or with partial overlap. It should be noted that the first coil units 11 and the second coil units 12 are electrically isolated (i.e., galvanically isolated). For example, an insulating material is provided between the first coil units 11 and the second coil units 12.

In an embodiment, the first region 110 is configured to be at least partially located within a radiation region. The radiation region is irradiated by radiation with a dose at or above a threshold during a radiation delivery. The second region 120 is configured to be at least partially located within a non-radiation region outside the radiation region. During the radiation delivery, a radiation beam passes through at least a portion of the first region 110.

In an embodiment, the first region 110 is configured to obtain a magnetic resonance imaging signal of a target site of the target object within the radiation region, and the second region 120 is configured to obtain a magnetic resonance imaging signal of a non-target site of the target object within the non-radiation region. The target site includes a region of interest of the target object, such as a target volume for radiotherapy. The non-target site refers to other sites other than the target site.

As an example, at least a portion of the at least one first coil unit 11 may be disposed in the radiation region. At least a portion of the at least one second coil unit 12 may be disposed in the non-radiation region. The radiation region may be at least a portion of a coverage region on the receiving coil device 10 that is covered by a radiation field generated by the radiotherapy apparatus during the radiation delivery. The non-radiation region may be a region on the receiving coil device 10 other than the radiation region. For example, during radiotherapy, the non-radiation region is not covered by the radiation field, or is covered by the radiation field but receives a radiation dose below a threshold. The radiation dose received by the non-radiation region during the radiation delivery is generally substantially lower than that of the radiation region. As an example, the threshold may be arbitrarily determined as needed (e.g., determined by computer calculation, determined manually, etc.), such as 0 Gy, 0.01 Gy, etc.

Optionally, the at least one first coil unit 11 may be entirely disposed in the radiation region on the receiving coil device 10. Optionally, one portion of the at least one first coil unit 11 may be disposed in the radiation region, and the other portion may be disposed in the non-radiation region.

Optionally, the at least one second coil unit 12 may be entirely disposed in the non-radiation region on the receiving coil device 10. Optionally, one portion of the at least one second coil unit 12 may be disposed in the non-radiation region, and the other portion may be disposed in the radiation region.

The first coil units 11 and the second coil units 12 are configured to receive magnetic resonance imaging signals. The magnetic resonance imaging signals are signals generated by the target object being excited by a transmission field provided by a transmitting coil in the magnetic resonance-guided radiotherapy apparatus.

By arranging the plurality of coil units in partitions, disposing the at least one first coil unit 11 in the radiation region and the at least one second coil unit 12 in the non-radiation region, the magnetic resonance imaging signals received by the first coil units 11 can be used for real-time imaging of the target region (e.g., the patient’s treatment site) in the radiation region. In addition, by disposing the second coil units 12 in the non-radiation region, imaging of regions other than the target region (e.g., a region within a specific range around the target region) can be performed, expanding the imaging range, thereby meeting the large-range imaging workflow requirements of the magnetic resonance-guided radiotherapy apparatus for pre-image guidance (e.g., adaptive radiation therapy (ART), image-guided radiation therapy (IGRT), etc.). The second coil units 12 in the non-radiation region are not affected by radiation or are only subject to relatively low interference, so the reliability of the magnetic resonance signal acquisition is high, achieving real-time auxiliary imaging of body sites of the patient located in the non-radiation region, providing more high-precision imaging results for radiotherapy, enhancing the imaging results, and thus improving the effectiveness of radiotherapy.

It should be noted that during the radiation delivery (e.g., radiotherapy, radiation verification, radiation processing) when magnetic resonance imaging is performed, radiation exposure may be performed simultaneously with magnetic resonance imaging. When the radiation exposure is performed simultaneously with the magnetic resonance imaging, the detailed anatomical structure of the site where the radiation acts and the immediate changes in tissue under the radiation exposure can be observed in real time. For example, when treating a tumor with radiotherapy, the physician can accurately determine the tumor’s location, boundary, and relationship with surrounding normal tissues based on clear images presented by the magnetic resonance imaging, and continuously monitor the tumor’s response to radiation, volume changes, etc., during the radiation exposure, thereby timely adjusting parameters such as radiation dose and irradiation angle based on real-time feedback, maximizing the therapeutic effect of radiotherapy on the tumor while better protecting the surrounding normal tissues from unnecessary radiation damage.

In an optional approach, during the radiation delivery (e.g., radiotherapy, radiation verification, radiation processing) when the magnetic resonance imaging is performed, the radiation exposure may be performed non-simultaneously with the magnetic resonance imaging (e.g., the magnetic resonance imaging is performed when no radiation is delivered).

When the radiation exposure is performed non-simultaneously with the magnetic resonance imaging, the possibility of mutual interference between devices under complex operating states is reduced, thus lowering the risk of device failure and extending the service life of the devices. Especially for some precision and expensive instrument components, additional losses caused by simultaneous operation are avoided, which can reduce the cost in device maintenance and replacement for medical institutions in the long run.

For example, in a treatment scenario, a patient may enter the device chamber along the main magnetic field direction of the magnetic resonance (corresponding to the B0 magnetic field direction) of the magnetic resonance-guided radiotherapy apparatus. When a site to be treated of the patient is the lungs, the patient’s lungs are located in the radiation region, and the patient’s head and shoulders are located in the non-radiation region. During operation of the apparatus, the transmitting coil excites the magnetic field, stimulating the patient to resonate. The first coil units 11 and the second coil units 12 can serve as imaging coil units, respectively used to acquire or detect magnetic resonance imaging signals, so that magnetic resonance images can be generated through subsequent steps such as analog-to-digital conversion, decoding, and digital signal processing. The imaging results corresponding to the first coil units 11 can reflect real-time conditions of the site to be treated. The first coil units 11 located in the radiation region may be affected by noise, making it difficult to accurately capture alternative respiratory monitoring signals, resulting in suboptimal imaging of the main tumor treatment region for alternative respiratory signal capture. The arrangement of the second coil units 12 can meet the respiratory signal capture requirement. The second coil units 12 are disposed in the non-radiation region for capturing a bulk motion of the patient, and the second coil units 12 can capture signals from regions of the patient not affected by respiration and heartbeat, such as the patient’s head and shoulders. When a change signal is acquired, the influence of respiration and heartbeat signals can be quickly ruled out, directly identifying the source of the image change as the bulk motion of the patient, allowing the physician to pause or adjust treatment immediately, thereby avoiding the radiation exposure to healthy tissues of the patient.

In an embodiment, when the displacement of the image of the patient’s site in the non-radiation region is detected to be greater than a preset displacement threshold, a fast scan sequence may be re-scanned to perform new magnetic resonance imaging, updating the structural item status in real time or updating the ground state impact acquisition.

The structural item status refers to various parameters or status information related to the patient’s body structure during monitoring, and the status information includes but is not limited to the following:
1. Position information: three-dimensional spatial coordinates of tissues or organs, used to determine whether abnormal displacement has occurred;
2. Morphological changes: whether the morphology of the tissues or organs has changed significantly, such as volume increase, volume decrease, or shape distortion; and
3. Motion state: dynamic parameters such as motion speed and acceleration of tissues or organs, used to assess the intensity of their motion.

The ground state impact acquisition and update refer to the process of reacquiring and updating the patient’s basic or reference state after abnormal bulk motion is detected, typically including:
1. Recalibration: recalibrating the magnetic resonance apparatus to ensure the accuracy of subsequent monitoring data;
2. Baseline data acquisition: reacquiring the patient’s basic physiological data, such as heart rate, blood pressure, respiratory rate, etc., to assess the impact of abnormal bulk motion on the patient’s overall physiological state; and
3. Image or data update: reacquiring image data to update the patient’s body structure images and assess the impact of abnormal bulk motion on the structural images.

Through the ground state impact acquisition and update, the system can more accurately assess the impact of abnormal bulk motion on the patient’s body and provide stronger support for subsequent treatment or intervention measures.

In an embodiment, the second region 120 is disposed on at least one side of the first region 110. Exemplarily, the second coil units 12 are disposed on at least one side of the first coil units 11.

For example, in the view of FIG. 1, the second coil units 12 may be disposed on at least one of a left side, a right side, an upper side, or a lower side of the first coil units 11. Specifically, the arrangement may be determined according to the treatment and monitoring requirements for various parts of the patient during radiotherapy.

For example, when performing radiotherapy and monitoring of a brain tumor in a patient, considering the need to precisely focus on the brain region while obtaining clear and comprehensive imaging of brain tissue, the second coil units 12 may be disposed on the lower side of the first coil units 11. While the first coil units 11 image the brain region, the second coil units 12 can image the chest region, allowing the physician to observe the specific morphology, location, and boundary with surrounding normal tissues of the brain tumor more accurately, as well as the patient’s motion, avoiding reduced effectiveness of the radiotherapy due to large displacement of the patient during treatment, and providing reliable data for subsequent precise radiotherapy.

Therefore, the second coil units 12 can be flexibly disposed on a side of the first coil units 11 according to different treatment and monitoring requirements for various parts of the patient during radiotherapy. This can fully leverage the synergy between the first coil units 11 and the second coil units 12, providing more effective imaging information to improve the accuracy of treatment, achieving better effectiveness of the radiotherapy, and ensuring a safer and more efficient treatment experience for the patient.

In an embodiment, the second coil units 12 may be disposed on at least one side of the first coil units 11 along the main magnetic field direction of the magnetic resonance (corresponding to the head-foot direction when the patient is positioned for treatment) of the magnetic resonance-guided radiotherapy apparatus. This arrangement can provide greater acceleration in the head-foot direction, making the magnetic field distribution more uniform in the longitudinal direction (head-foot direction) of the human body, thereby reducing image artifacts caused by inhomogeneity of the magnetic field, allowing physicians to see more realistic and clearer images of human tissues, whether observing the morphology and structure of tumor tissues or surrounding normal tissues, more accurately, so as to facilitate precisely determining the lesion range and formulating subsequent appropriate radiotherapy strategies.

Furthermore, the human body has different distributions of tissues and organs in the head-foot direction, with a relatively large length range. This arrangement can better expand the magnetic field coverage according to the morphological characteristics of the human body in the head-foot direction, ensuring that key sites from the head to the feet are within the effective magnetic field region, thus making the device applicable to most treatment scenarios and improving its versatility. In addition, this arrangement can avoid situations where the magnetic field is too weak in some regions, which affects the imaging and treatment effect. Especially for some radiotherapy and monitoring needs involving whole-body scans or long-segment lesions (such as tumors in the spinal region), this arrangement can provide good magnetic field support.

In an embodiment, the receiving coil device 10 includes: at least one row of first coil units 11 located in the first region 110, and at least one row of second coil units 12 located in the second region 120.

The at least one row of first coil units 11 may be disposed on a first side. The at least one row of second coil units 12 may be disposed on a second side opposite to the first side. As shown in FIG. 1, the first side may refer to one side of the receiving coil device 10 (e.g., the upper side shown in FIG. 1), and the second side may refer to the other side of the receiving coil device 10 (e.g., the lower side shown in FIG. 1).

The specific number of rows of the first coil units 11 and the second coil units 12 may be arbitrarily set according to the treatment scenario.

In an optional implementation, one side of the receiving coil device 10 corresponds to a head direction (or a foot direction) of the magnetic resonance-guided radiotherapy apparatus, and the other side of the receiving coil device 10 corresponds to the foot direction (or the head direction) of the magnetic resonance-guided radiotherapy apparatus, which is not limited in the present disclosure. For example, one side of the receiving coil device 10 may also correspond to the left side (or right side) of a human body lying on a sickbed, and the other side of the receiving coil device 10 may correspond to the right side (or left side) of the human body.

By disposing at least one row of first coil units 11 and at least one row of second coil units 12 in the second direction corresponding to the head-foot direction, greater acceleration in the head-foot direction can be achieved. The more magnetic resonance imaging signals obtained in the head-foot direction, the faster the imaging speed of the radiation region, which is beneficial for improving the real-time imaging speed of the main treatment region of the patient during treatment. The faster the real-time imaging speed, the faster the acquisition of actual changes in the patient’s site to be treated, which is beneficial for the physician to timely control radiotherapy based on actual conditions during the radiotherapy.

In an embodiment, as shown in FIG. 1, the second coil units 12 are distributed on one side of the first coil units 11 along the second direction. The first coil units 11 and the second coil units 12 may be arranged in an array as shown in FIG. 1, but are not limited thereto, and may also be arranged in other manners, such as the first coil units 11 and the second coil units 12 being arranged side by side in elongated strips.

As shown in FIG. 1, the second coil units 12 are distributed on one side of the first coil units 11 along the second direction (corresponding to the head-foot direction of the human body). When positioning the patient, the patient can be positioned according to the location of the site to be treated and the site to be monitored outside the radiation region. For example, if the patient’s lungs need to be treated and the movement of the patient’s shoulders needs to be monitored, the patient’s head can be oriented toward the direction of the second coil units 12 to enter the region of the receiving coil device 10, so that the shoulders are located in the non-radiation region and the lungs are located in the radiation region. By disposing the second coil units 12 on one side of the first coil units 11, monitoring of both the patient’s treatment site and non-treatment site can be achieved with a small-volume coil design.

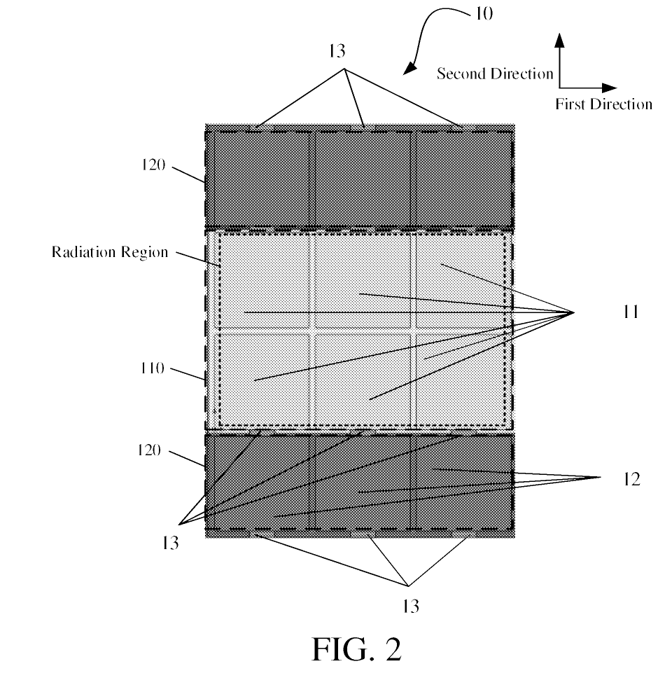

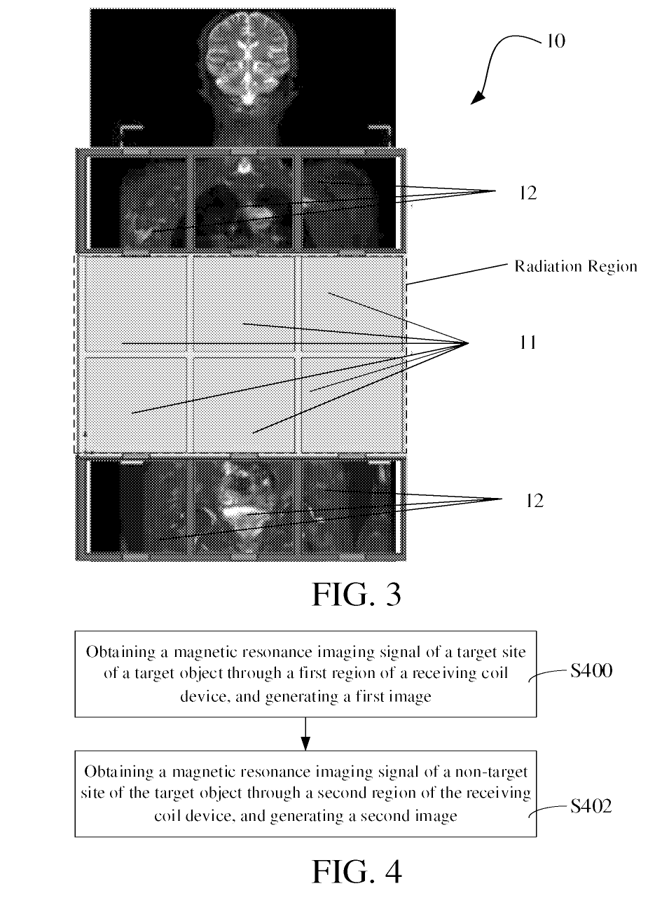

In an embodiment, as shown in FIGS. 2 and 3, the receiving coil device 10 includes: a first row of second coil units 12, a second row of second coil units 12, and at least one row of first coil units 11.

The first row of second coil units 12 may be disposed on a first side, the second row of second coil units 12 may be disposed on a second side opposite to the first side, and the at least one row of first coil units 11 is disposed between the first row of second coil units 12 and the second row of second coil units 12. As shown in FIG. 2, the first side may refer to one side of the receiving coil device 10 (e.g., the upper or lower side in FIG. 2), the second row of second coil units 12 is disposed on the other side of the receiving coil device 10 (e.g., the lower or upper side in FIG. 2), and the at least one row of first coil units 11 is disposed between the first row of second coil units 12 and the second row of second coil units 12.

An example where the first row of second coil units 12 and the second row of second coil units 12 are distributed on two sides of the first coil units 11 along the second direction (i.e., the head-foot direction of the human body) will be described. As shown in FIG. 3, if the patient’s lungs need to be treated and the movement of the patient’s shoulders needs to be monitored, the patient’s head can enter the region of the receiving coil device 10 from either side of the first coil unit 11 along the second direction, so that the shoulders are located in the non-radiation region, making patient positioning more flexible.

In an embodiment, each coil unit corresponds to one channel for magnetic resonance imaging. Compared to the single-channel configuration of the receiving coil device in the main magnetic field direction of the magnetic resonance in the related art, the receiving coil device 10 of the present disclosure forms a plurality of channels for magnetic resonance imaging in the main magnetic field direction of the magnetic resonance (i.e., the second direction). As shown in FIG. 1, a 3x3 array of coil units forms nine channels for magnetic resonance imaging. Whether in the direction of the main magnetic field of magnetic resonance or in the direction perpendicular to the main magnetic field, i.e., the longitudinal direction or the transverse direction of the human body, the receiving coil device 10 of the present disclosure has a plurality of channels for magnetic resonance imaging. Each row of coil units receives signals through its dedicated channel, allowing more effective magnetic resonance imaging signals to be gathered, thereby enhancing signal strength overall. In addition, with a multi-channel configuration, magnetic resonance imaging signals can be acquired in parallel. Different rows of coil units can operate simultaneously, and acquire signals from multiple sites or different layers of tissue within the same time, greatly shortening the overall scanning time and improving the imaging efficiency.

To meet the coverage requirement of a large imaging region with coil units, the size of each row of first coil units 11 should not be too large. This is because an excessively large single-channel coil unit may affect data acquisition speed. Moreover, an excessively large single-channel coil unit may limit the number of channels and a coverage region within the target region, reducing the imaging efficiency. An excessively large single-channel coil unit may also make it difficult to maintain uniform radio frequency excitation in the main magnetic field direction of the magnetic resonance, potentially introducing local inhomogeneity of the magnetic field and affecting the image quality. Therefore, the size of a single row of first coil units 11 can be set according to requirements for the imaging efficiency, image quality, etc.

In an embodiment, each row of the at least one row of first coil units 11 includes one or more first coil units 11 arranged side by side, and/or each row of the at least one row of second coil units 12 includes one or more second coil units 12 arranged side by side.

Optionally, each row of the at least one row of first coil units 11 includes a plurality of first coil units 11 arranged side by side, and/or each row of the at least one row of second coil units 12 includes a plurality of second coil units 12 arranged side by side. Moreover, two adjacent first coil units 11 are electromagnetically coupled, and two adjacent second coil units 12 are electromagnetically coupled.

By providing the plurality of coil units arranged side by side in each row, the coverage of a wider region in a row direction can be achieved. For example, when performing magnetic resonance imaging on a human body, whether for the torso or limbs, the increased number of coil units in the row direction can receive magnetic resonance imaging signals from human tissues over a wider range, avoiding missing signals from some tissues due to limited coverage of a single coil unit, thereby obtaining tissue information at different depths and locations in the human body more comprehensively, and providing a richer data foundation for generating complete and accurate magnetic resonance images.

In an embodiment, as shown in FIGS. 1 to 3, each row of the at least one row of first coil units 11 includes a plurality of first coil units 11 arranged side by side, and a column direction is the second direction or the head-foot direction.

By arranging multiple rows of first coil units 11 along the second direction, a large range of the magnetic resonance imaging can be achieved. Specifically, the arrangement of the multiple rows of first coil units 11 can be determined by weighing factors such as a superficial signal-to-noise ratio, a depth signal-to-noise ratio of the receiving coil, and an overall parallel imaging acceleration capability of the receiving coil device 10 in the actual application scenario.

In an embodiment, as shown in FIGS. 2 to 3, each row of the at least one row of second coil units 12 includes a plurality of second coil units 12 arranged side by side, and the column direction is the second direction or the head-foot direction.

Similar to the multiple-row arrangement of the first coil units 11, the second coil units 12 may also be arranged in multiple rows, and the multiple rows of second coil units 12 are arranged along the second direction. This arrangement can increase the imaging range of the patient’s sites in the non-radiation region, providing more imaging references for radiotherapy. The arrangement of the multiple rows of second coil units 12 can also be determined by the weighing factors such as the superficial signal-to-noise ratio, the depth signal-to-noise ratio of the receiving coil, and the overall parallel imaging acceleration capability of the receiving coil device 10 in the actual application scenario.

In an embodiment, the overall contour of the first coil units 11 matches the shape of the radiation region. In the case of shape matching, it is beneficial for accurately obtaining magnetic resonance imaging results of the radiation region, and also beneficial for arranging the second coil units 12 close to the first coil units 11, so that the second coil units 12 can enhance the magnetic resonance imaging of the first coil units 11, improving the imaging quality of the radiation region.

For example, when there are multiple rows of first coil units 11, the lengths of the first coil units 11 in different rows may be the same or different. When there are multiple columns of first coil units 11, the lengths of the first coil units 11 in different columns may be the same or different. Specifically, the arrangement can be set according to the shape of the radiation region. For example, if the radiation region is circular, the plurality of first coil units 11 may be arranged to form a generally circular shape.

Similarly, when there are multiple rows of second coil units 12, the lengths of the second coil units 12 in different rows may be the same or different. When there are multiple columns of second coil units 12, the lengths of the second coil units 12 in different columns may be the same or different. Specifically, the arrangement can be set according to the shape of the non-radiation region.

In an embodiment, as shown in FIGS. 1 to 3, the receiving coil device 10 further includes one or more processing circuits connected to the plurality of coil units in a one-to-one correspondence, i.e., each coil unit is connected to one processing circuit. The one or more processing circuits 13 are disposed in the non-radiation region. Each processing circuit includes one or more electronic components 13. In some embodiments, the processing circuit is an electronic component 13. In some embodiments, the processing circuit includes other circuit components (e.g., leads, other components, etc.) in addition to the electronic component.

Exemplarily, the receiving coil device 10 includes at least one first coil unit, at least one second coil unit, and at least one electronic component. Each of the at least one electronic component includes a capacitor, an inductor, or an operational amplifier, etc. The at least one electronic component is connected to the at least one first coil unit and/or the at least one second coil unit. Exemplarily, the at least one electronic component is disposed in the non-radiation region. In a possible configuration, the electronic component is disposed between the first region 110 and the second region 120.

The processing circuit refers to a circuit capable of supporting the coil unit in receiving and processing magnetic resonance imaging signals for imaging. For example, the processing circuit can include a resonant circuit that supports tuning the coil unit to operate at a resonant frequency corresponding to the magnetic resonance imaging signals, thereby achieving reception of the magnetic resonance imaging signals. The processing circuit may further include a signal processing circuit. The signal processing circuit supports signal processing such as amplification of the magnetic resonance imaging signals received by the coil units to generate clearer images. Optionally, each processing circuit can include a plurality of electronic components such as a capacitor, an inductor, and an operational amplifier, etc. Each processing circuit includes at least some components sensitive to radiation.

Specifically, by arranging the plurality of coil units in partitions, arranging the first coil units 11 in the radiation region of the radiotherapy apparatus, arranging the second coil units 12 in the non-radiation region of the radiotherapy apparatus, and arranging the processing circuits 13 in the non-radiation region, the magnetic resonance imaging signals received by the first coil units 11 can be used for real-time imaging of the patient’s treatment site in the radiation region. The radiation region has no processing circuits 13 and no radiation-sensitive or complex electronic components, ensuring the imaging reliability and accuracy of real-time images of the radiation region achieved based on the first coil units 11. In addition, arranging the second coil units 12 in the non-radiation region can meet the large-range imaging workflow requirements. The second coil units 12 and the processing circuits 13 in the non-radiation region are not affected by radiation, so the reliability of their magnetic resonance signal acquisition is high, achieving real-time auxiliary imaging of the patient’s non-radiation region sites, providing more high-precision imaging results for radiotherapy, thereby improving the effectiveness of radiotherapy.

By disposing the processing circuits 13 outside the radiation range, the processing circuits 13 can be prevented from being affected by the radiation beam during radiotherapy. Therefore, the accuracy of tumor radiotherapy planning and dose calculation can be ensured, and the effectiveness of radiotherapy can be improved.

The first coil units 11 and the second coil units 12 are connected to different processing circuits to correspond to different imaging channels. With a greater number of coil units, more imaging channels can be provided, which is beneficial for improving the imaging speed and imaging quality.

The receiving coil device 10 provided in the embodiments of the present disclosure, based on the design of a small-size channel of the coil, can ensure that no sensitive or complex electronic components are present in the radiation region requiring real-time imaging, and ensure the real-time imaging quality in the non-radiation region.

In other embodiments, the present disclosure further provides a receiving coil device 10 applied to magnetic resonance-guided radiation delivery. The receiving coil device 10 includes a first region. The first region includes at least one first coil unit 11 arranged along a first direction, and includes at least two rows of first coil units 11 in a second direction. The first direction is perpendicular to the second direction.

In an embodiment, the receiving coil device further includes an electronic component connected to the first coil unit. The electronic component is not disposed between adjacent rows of first coil units in the second direction, i.e., the first coil units in the first region do not have electronic components placed between them, which can ensure that there are no radiation-sensitive electronic components in the radiation region for real-time imaging.

In an embodiment, the receiving coil device further includes a second region disposed on at least one side of the first region. For example, the second region is disposed on at least one side of the first region along the second direction, and the second region includes at least one row of second coil units.

The electronic component connected to the coil unit located in the first region is disposed in a region on at least one side of the first region along the second direction. As shown in FIG. 1, the electronic components 13 connected to the coil units located in the first region are disposed in the regions on both sides of the first region along the second direction, i.e., in the regions of the upper and lower sides of the first region. The electronic component connected to the coil unit located in the second region is disposed in a region on a side of the second region away from the first region along the second direction. As shown in FIG. 1, the electronic components 13 connected to the coil units located in the second region are disposed in the lower region of the second region along the second direction.

In other embodiments, the present disclosure further provides a receiving coil device 10 applied to magnetic resonance-guided radiation delivery. The receiving coil device 10 includes a first region 110, a second region 120, and an electronic component. The first region 110 includes at least one first coil unit 11 arranged along a first direction, and the second region 120 includes at least one second coil unit 12 arranged along the first direction. The electronic component is disposed in a region other than the first region 110 and is electrically connected to the at least one first coil unit 11 and/or the at least one second coil unit 12. In an embodiment, the at least one first coil unit 11 is electrically isolated from the at least one second coil unit 12. The electronic component is disposed in the region other than the first region, i.e., in the region other than the radiation region, which ensures that there are no radiation-sensitive electronic components in the radiation region for real-time imaging. For example, the electronic component may be disposed in the second region, or in a region other than the first and second regions.

Exemplarily, as shown in FIG. 1, the first region 110 and the second region 120 are arranged along a second direction, and the first direction is perpendicular to the second direction. The second direction may be set as a main magnetic field direction (i.e., the head-foot direction of the human body, also referred to as the longitudinal direction of the human body) of the magnetic resonance-guided radiotherapy apparatus, and the first direction may be set as a direction (i.e., a direction perpendicular to the head-foot direction, also referred to as the transverse direction of the human body) perpendicular to the main magnetic field direction of the radiotherapy apparatus.

Exemplarily, the first region 110 is configured to be at least partially located within a radiation region. The radiation region is irradiated by radiation with a dose at or above a threshold during a radiation delivery. The second region 120 is configured to be at least partially located within a non-radiation region outside the radiation region.

Exemplarily, during the execution of the radiation delivery, at least a portion of the first region 110 is positioned within a radiation field of a radiation delivery apparatus, and the second region 120 is positioned outside the radiation field, i.e., the first coil units are within the radiation field formed by the radiation beam, and the second coil units are outside the radiation field.

In an embodiment, the at least one first coil unit has a lower material radiation attenuation than the at least one second coil unit. In another embodiment, the at least one first coil unit has a higher material density uniformity than the at least one second coil unit.

In an embodiment, as shown in FIGS. 1 to 3, the electronic components 13 connected to the first coil units 11 located in the first region 110 are disposed on sides of the first coil units 11 that are close to the second region 120, which is beneficial for reducing the routing length between the first coil units 11 and the electronic components 13, thereby reducing routing costs, and also beneficial for improving the compactness of the coil units, avoiding magnetic resonance imaging blind spots caused by excessive gaps between the plurality of coil units.

In an embodiment, as shown in FIGS. 1 to 3, the electronic components 13 connected to the second coil units 12 located in the second region 120 are disposed on sides of the second coil units 12 that are away from the first region 110. By disposing the electronic components 13 connected to the second coil units 12 and the electronic components 13 connected to the first coil units 11 on two sides of the second coil units 12, it is beneficial for isolating the electronic components 13, avoiding device interference between the electronic components 13, and also preventing excessive gaps between the first coil units 11 and the second coil units 12 due to too many electronic components 13 at the adjacent positions of the first coil units 11 and the second coil units 12, which would generate the magnetic resonance imaging blind spots.

In an embodiment, the receiving coil device 10 includes a plurality of first coil units 11, and every two adjacent first coil units 11 are electromagnetically coupled.

The first coil units 11 are used for real-time imaging of the patient’s site in the radiation region. The electromagnetic coupling between the plurality of first coil units 11 can ensure blind-spot-free imaging of the patient’s site in the radiation region, providing comprehensive and effective real-time imaging for the radiotherapy process.

Optionally, the arrangement of the plurality of first coil units 11 may be hexagonal, circular, or other designs, with the principle of improving the uniformity of distribution. The radiation regions of the first coil units 11 of two adjacent channels at least partially overlap to avoid blind spots.

In an embodiment, the receiving coil device 10 includes a plurality of second coil units 12, and every two adjacent second coil units 12 are electromagnetically coupled. This can ensure blind-spot-free imaging of the patient’s site in the non-radiation region, facilitating effective monitoring of the patient’s motion in the non-radiation region by the physician.

In an embodiment, the first coil units 11 may adopt a lower-impedance, thinner antenna design.

In an embodiment, the receiving coil device 10 may use a flexible low-density material for external mechanical design while ensuring the overall support strength of the coils.

In an embodiment, a radio frequency system is provided. The radio frequency system includes the receiving coil device 10 as described in any one of the above embodiments.

The radio frequency system, in addition to including the receiving coil device 10, further includes a transmitting coil and a signal source. The signal source excites the transmitting coil (not shown) to transmit radio frequency signals, stimulating the human body to resonate. The receiving coil device 10 receives magnetic resonance imaging signals generated during the resonance of the human body, and transmits them to the processing circuits 13. The processing circuits 13 can perform analog-to-digital conversion, decoding, digital signal processing, etc. on the received magnetic resonance imaging signals to generate magnetic resonance images.

The radio frequency system equipped with the above-described receiving coil device 10, due to the partitioned arrangement of the coil units, can ensure the reliability of the real-time imaging in the radiation region and also monitor of the patient’s motion in the non-radiation region, providing the physician with real-time high-quality images of the main treatment region and monitoring images of the patient’s bulk motion during radiotherapy, thereby improving the effectiveness of radiotherapy.

In an embodiment, a magnetic resonance-guided radiotherapy apparatus is provided, including the receiving coil device 10 as described in any one of the above embodiments.

During radiotherapy, the magnetic resonance-guided radiotherapy apparatus equipped with the above-described receiving coil device 10 can provide real-time magnetic resonance imaging of the patient’s site in the radiation region, and magnetic resonance imaging of the patient’s site in the non-radiation region for imaging enhancement, ensuring the quality of the magnetic resonance imaging in both regions, and providing high-precision, highly reliable reference images for the radiotherapy process, thereby improving the accuracy of the radiotherapy.

Furthermore, the processing circuits 13 are disposed in the non-radiation region, which avoids affecting the radiation beam during radiotherapy, thereby improving the accuracy of tumor radiotherapy planning and dose calculation, and improving the effectiveness of radiotherapy.

Based on the increased imaging channels of the second coil units 12, abnormal motion monitoring of the patient’s sites in the non-radiation region can be performed. Based on a monitoring result, when an image displacement of the monitored site such as the shoulders is greater than a preset displacement threshold, it is determined that the patient has experienced abnormal motion. The preset displacement threshold may be set according to the requirement for the displacement of the patient during radiotherapy.

In an embodiment, the magnetic resonance-guided radiotherapy apparatus further includes a display device (not shown). When the image displacement of the monitored site is greater than the preset displacement threshold, the processing circuit sends reminder content such as “abnormal motion reminder” to the display device for display, to remind the physician to pause the radiotherapy in a timely manner.

In an embodiment, when the image displacement of the monitored site is greater than the preset displacement threshold, the magnetic resonance-guided radiotherapy apparatus may re-scan a fast scan sequence to perform new magnetic resonance imaging, and compare the new magnetic resonance image with the magnetic resonance image from the pre-scan stage. If they match, it indicates that the patient has not experienced abnormal motion. If they do not match, it indicates that the patient has indeed experienced abnormal motion. In this case, the reminder content such as “abnormal motion reminder” is displayed on the display device.

In an embodiment, the present disclosure further provides a magnetic resonance-guided radiation delivery method, as shown in FIG. 4, including: S400, obtaining a magnetic resonance imaging signal of a target site of a target object through a first region 110 of a receiving coil device, and generating a first image; S402, obtaining a magnetic resonance imaging signal of a non-target site of the target object through a second region 120 of the receiving coil device, and generating a second image. The first image is configured for radiation delivery, and the second image is configured for monitoring the target object.

The receiving coil device may be any of the receiving coil devices described in the above embodiments. The target object refers to a patient, a phantom, etc., requiring the radiation delivery. The target site includes a region of interest of the target object, such as a target volume of radiotherapy. The non-target site refers to other sites other than the target site, such as a volume within a specific range around the target volume.

Exemplarily, magnetic resonance imaging signals of a target volume of tumor of the patient can be obtained in real time through at least one first coil unit in the first region 110 of the receiving coil device. The magnetic resonance imaging signals are subjected to subsequent processing steps such as analog-to-digital conversion, decoding, and digital signal processing to generate a magnetic resonance image, i.e., the first image. The first image can be configured to monitor in real time the response of the target volume to radiation, volume changes of the target volume, etc., thereby facilitating the operator to timely adjust parameters such as radiation dose and irradiation angle.

Exemplarily, when performing radiation delivery on a brain tumor in a patient, to avoid reduced radiotherapy effectiveness due to a large displacement of the patient during treatment, magnetic resonance imaging signals of the patient’s chest region can be obtained through at least one second coil unit in the second region 120 of the receiving coil device. The magnetic resonance imaging signals of the chest region are processed to generate a magnetic resonance image, i.e., the second image. The second image can be configured to monitor the patient’s motion in real time, for example, to monitor the patient’s abnormal motion and/or normal motion. In an example, the second image can be configured to capture a respiratory signal of the patient. In another example, the second image can be configured to capture signals from regions of the patient not affected by respiration and heartbeat, such as the patient’s head and shoulders. When a change signal appears in the second image, the influence of respiration and heartbeat signals can be quickly ruled out, directly identifying the source of the image change as the abnormal motion of the patient.

Specifically, the displacement of the non-target site, such as the shoulder, in the second image can be detected. When the image displacement of the monitored site such as the shoulder is greater than a preset displacement threshold, it is determined that the patient has experienced abnormal motion. The preset displacement threshold may be set according to the requirement for the displacement of the patient during radiotherapy. Further, when abnormal motion of the patient is detected, a first response operation is triggered. For example, pausing radiation delivery, adjusting radiation beam parameters, updating imaging, and/or generating reminder information.

In other examples, the second image may also include an image of the target site (target volume). For example, for radiotherapy and monitoring needs involving long-segment lesions (such as tumors in the spinal region), the second image can serve as an extended image of the first image, allowing the operator to observe the morphology and structure of the tumor tissue and surrounding normal tissues over a large range, and providing more effective imaging information to improve the treatment accuracy.

It should be understood that steps S400 and S402 may be performed at least partially simultaneously or sequentially, and the present disclosure does not limit the order of execution.

The magnetic resonance-guided radiation delivery method provided by the present disclosure can maximize the therapeutic effect of radiotherapy on tumors based on the first image, while better protecting surrounding normal tissues from unnecessary radiation damage based on the second image.

In an embodiment, the present disclosure further provides an image display method, applied to magnetic resonance-guided radiation delivery. The image display method includes: displaying a first image to a user during a first period within the radiation delivery; and displaying a second image to a user during a second period within the radiation delivery, before the radiation delivery, and/or after the radiation delivery.

The first image includes a target region of the radiation delivery, and the second image includes a non-target region of the radiation delivery. In an example, the target region refers to a region (e.g., a target volume of tumor) where a target site of the target object (e.g., patient) is located, which is also the region located within the radiation region. The non-target region refers to a region where a non-target site of the target object is located, which is also the region located outside the radiation region, such as the region around the target volume. It should be noted that the first image does not include the non-target region.

During the second period within the radiation delivery, the second image may be switched to display the first image, or the first image and the second image may be displayed simultaneously, facilitating the operator to observe real-time conditions of both the target region and the non-target region. Displaying the second image before the radiation delivery allows confirmation of the patient’s state before the radiotherapy. Displaying the second image after the radiation delivery allows monitoring of whether the patient has any abnormalities after the radiotherapy.

The image display method provided by the present disclosure, by displaying the real-time magnetic resonance image of the patient’s target site and displaying the magnetic resonance image of the patient’s non-target site, broadens the imaging range, and provides high-precision, highly reliable reference images for the radiotherapy process, thereby improving the accuracy of the radiotherapy.

In an embodiment, the present disclosure further provides a non-transitory computer-readable storage medium having a computer program stored thereon. The computer program, when executed by a processor, implements the steps of any of the above method embodiments.

In the description of this specification, reference to the description of the terms “some embodiments”, “other embodiments”, etc. means that a particular feature, structure, material or feature described in connection with the embodiment or example is included in the present specification at least one embodiment or example of the application. In this specification, schematic descriptions of the above terms do not necessarily refer to the same embodiment or example.

The technical features in the above embodiments may be combined arbitrarily. For concise description, not all possible combinations of the technical features in the above embodiments are described. However, provided that they do not conflict with each other, all combinations of the technical features are to be considered to be within the scope of protection of the present disclosure.

The above-mentioned embodiments only describe several implementations of the present disclosure, and their description is specific and detailed, but should not be understood as a limitation on the protection scope of the present disclosure. It should be noted that, for a person of ordinary skill in the art, various variations and improvements can be further made without departing from the conception of the present disclosure, and these all fall within the protection scope of the present disclosure. Therefore, the protection scope of the present disclosure should be subject to the appended claims.

What is claimed is:

1. A receiving coil device, applicable to magnetic resonance-guided radiation delivery, the receiving coil device comprising:
a first region comprising at least one first coil unit arranged along a first direction;
a second region comprising at least one second coil unit arranged along the first direction; and
an electronic component disposed in a region other than the first region and electrically connected to the at least one first coil unit and/or the at least one second coil unit.

2. The receiving coil device according to claim 1, wherein the first region and the second region are arranged along a second direction, and the first direction is perpendicular to the second direction.

3. The receiving coil device according to claim 1, wherein the at least one first coil unit is electrically isolated from the at least one second coil unit.

4. The receiving coil device according to claim 1, wherein the at least one first coil unit has a lower material radiation attenuation than the at least one second coil unit, and/or the at least one first coil unit has a higher material density uniformity than the at least one second coil unit.

5. The receiving coil device according to claim 1, wherein the first region is configured to be at least partially located within a radiation region, the radiation region being irradiated by radiation with a dose at or above a threshold during a radiation delivery, and the second region is configured to be at least partially located within a non-radiation region outside the radiation region.

6. The receiving coil device according to claim 2, wherein the first region comprises at least two rows of first coil units arranged along the second direction; and
the electronic component connected to the coil unit located in the first region is disposed in a region on at least one side of the first region along the second direction, and the electronic component connected to the coil unit located in the second region is disposed in a region on a side of the second region away from the first region along the second direction.

7. A receiving coil device, applicable to magnetic resonance-guided radiation delivery, the receiving coil device comprising:
a first region comprising at least two rows of first coil units, each row of first coil units comprising a plurality of coil units arranged along a first direction, the at least two rows of first coil units being arranged along a second direction, and the first direction being perpendicular to the second direction; and
an electronic component not disposed between adjacent rows of first coil units along the second direction.

8. The receiving coil device according to claim 7, further comprising:
a second region disposed on at least one side of the first region, the second region comprising at least one row of second coil units;
wherein the electronic component connected to the coil unit located in the first region is disposed in a region on at least one side of the first region along the second direction, and the electronic component connected to the coil unit located in the second region is disposed in a region on a side of the second region away from the first region along the second direction.

9. A receiving coil device, applicable to magnetic resonance-guided radiation delivery, the receiving coil device comprising:
a plurality of coil units, comprising at least one first coil unit in a first region and at least one second coil unit in a second region,
wherein the at least one first coil unit is arranged along a first direction, and the at least one second coil unit is arranged along the first direction; and
wherein the first region and the second region are arranged along a second direction, and the first direction is perpendicular to the second direction.

10. The receiving coil device according to claim 9, wherein the first region is configured to be at least partially located within a radiation region, the radiation region being irradiated by radiation with a dose at or above a threshold during a radiation delivery, and the second region is configured to be at least partially located within a non-radiation region outside the radiation region.

11. The receiving coil device according to claim 9, wherein the receiving coil device further comprises an electronic component disposed in the non-radiation region, the electronic component being connected to the at least one first coil unit and/or the at least one second coil unit.

12. The receiving coil device according to claim 9, wherein the second region is disposed on at least one side of the first region along the second direction.

13. The receiving coil device according to claim 9, wherein:
at least one row of first coil units located in the first region is disposed on a first side of the receiving coil device; and
at least one row of second coil units located in the second region is disposed on a second side opposite to the first side of the receiving coil device in the second direction.

14. The receiving coil device according to claim 9, wherein:
a first row of second coil units located in the second region is disposed on a first side of the receiving coil device;
a second row of second coil units located in the second region is disposed on a second side opposite to the first side of the receiving coil device in the second direction; and
at least one row of first coil units located in the first region is disposed between the first row of second coil units and the second row of second coil units.

15. The receiving coil device according to claim 14, wherein each row of the at least one row of first coil units comprises one or more first coil units arranged side by side, and/or each row of the at least one row of second coil units comprises one or more second coil units arranged side by side.

16. The receiving coil device according to claim 9, wherein a plurality of channels for magnetic resonance imaging are formed in the second direction.

17. The receiving coil device according to claim 9, wherein the plurality of coil units comprise a plurality of first coil units located in the first region and a plurality of second coil units located in the second region, two adjacent first coil units are electromagnetically coupled, and two adjacent second coil units are electromagnetically coupled.

18. The receiving coil device according to claim 11, wherein the electronic component connected to the coil unit located in the first region is disposed on a side of the coil unit located in the first region that is close to the second region, and the electronic component connected to the coil unit located in the second region is disposed on a side of the coil unit located in the second region that is away from the first region.

19. The receiving coil device according to claim 10, wherein the first region is configured to obtain a magnetic resonance signal of a target site of a target object within the radiation region, and the second region is configured to obtain a magnetic resonance signal of a non-target site of the target object within the non-radiation region.

20. A magnetic resonance-guided radiotherapy apparatus, comprising the receiving coil device according to claim 1.
